(19) United States
(12) Patent Application Publication
(10) Pub. No.: US 2002/0168423 A1
(43) Pub. Date: Nov. 14, 2002

(54) High strength liquid antiseptic

(76) Inventor: Stephen Ray Wurzburger, Goodyear's Bar, CA (US)

Correspondence Address: ROBERT SAMUEL SMITH, 1263 EMORY STREET, SAN JOSE, CA, 95126, US

(21) Appl. No.: 10/021,793

(22) Filed: Dec. 12, 2001

Related U.S. Application Data
(63) Continuation-in-part of application No. 09/307,100, filed on May 7, 1999, now Pat. No. 6,331,514.
Application No. 09/307,100 is a continuation-in-part of application No. 08/994,547, filed on Dec. 19, 1997, now abandoned.

(51) Int. Cl. A61K 33/04

(52) U.S. Cl. 424/709

(57) ABSTRACT

A disinfectant using as a base formed by selecting one of a metal, metal hydride, metal oxide metal hydroxide and an acid wherein the acid forms an insoluble salt with the metal, adding to a solution of the acid in gram equivalent amounts the metal hydride and filtering the resultant solution to obtain a form of essentially pure activated water that exhibits a high oxidation potential as evidenced by a pH less than 2. Analysis of the activated water shows less than 2500 ppm sulfate concentration which is cetainly substatnially less than a concetration to explain the low pH. In order to adapt the “activated water” to application as an antiseptic to the skin, a carrier (film forming oil) is added which forms a film and retains metal salts that render the remaining film bactericidal after the activated water has evaporated.

### CROSS REFERENCE TO EARLIER FILED APPLICATIONS

This application is a continuation-in part of application Ser. No. 09/307,100 filed May 07, 1999 which is a continuation-inpart of application Ser. No. 08-994,547 filed Nov. 19, 1997 from which priority is claimed.

### FIELD OF THE INVENTION

This application relates to antiseptic compounds and to an aqueous solution particularly suited for application to the skin that provides a greater than 99.99 % microbial efficacy with a single application.

### BACKGROUND AND INFORMATION DISCOSURE

Many different substances can be classified as disinfectants. Chemical agents acting as disinfectants include strong acids and bases. The modes of action include five general types: oxidation, hydrolysis, modification of cell membrane permeability, mechanical disruption, chemical union.

A number of sulfur containing pharmaceutical compounds have been disclosed for use as bactericides.

For example, U.S. Pat. No. 4,006,251 to Taylor et al discloses Thiocarbamylsulfenamide An apparatus (MicroWater™ distributed by Optimum Health Institute, San Mateo, Calif.) has been disclosed. The device produces two kinds of water with different redox potentials, one with a high reduction potential (referred to as “alkaline MicroWater”) and one with a high oxidation potential (referred to as “acid MicroWater”).

The acid Microwater has a been found to have commercially viable bactericidal properties when used in the lowest pH range (2.5) attainable (reported) for this solution. It is believed that the active molecule is the “hydronium” ion having the structure:

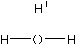

Two excellent references on the subject of free radicals are found in the books:

“Excited States and Free Radicals in Biology and Medicine” by Bensassoon and Land (Oxford Science Publications

“The Oxygen Paradox” by Davies and Ursini (CLEEUP University Press.)

The desire to express germicidal activity of various agents quantitatively has led to the development of numerous procedures, most of which are based on a phenol coefficient method developed by Rideal and Walker in 1906. The method involves culturing two batches of selected microbes for a period of time, one batch is disposed in a “standard” bactericide and the other batch is disposed in the “test” bactericide. A quantitative expression of effectiveness of the test bactericide may be expressed as a percent of the number of bacteria killed by test bactericide compared to the standard bactericide. Standard tests are outlined under ASTM guidelines.

Liquid compositions used as antiseptics for health care workers, personnel involved with food preparation and the traveling public present a unique set of problems in terms of skin sensitivity including allergic reactions that vary among members of the public. These compositions are intended to prevent transmission of potentially pathogenic microorganisms, particularly via the hands. Credible evidence has been discovered that recontamination of the skin suffice often occurs by using paper towels that have been used by the previous user. It has been discovered that towel material can be contaminated at the time of manufacture or contamination during installation in its dispenser.

There are numerous patents and scientific artiles that are related to anti-microbial disinfectants that may be considered for personal and antiseptic purposes.

A number of these references describe the relative merits of various germicidal substances including quaternary ammonium compounds, alcohols, ketones, chlorhexidines, iodophors, hexachlorophenes, proprolactinos,organic acids, sulfur compounds, glycols. and heavy metal salts. Published research has established the general range of concentrations of each substance that can be applied to generate the greatest log kill over a large variety of bacteria, viruses and fungi and still be safe for multiple applications during a short period. The chemical compositions of a majority of these antiseptic compounds are very similar. Typically, they are composed of 50 to 90 % alcohol, usually methanol, isopropanol or a mixture thereof. The remaining compound is water and sometimes there will be a small percentage of a secondary cationic germicide, such as a quaternary ammonium compound, hexachloraphenes of a metal salt together with a moisturizer and some sort of a fragrant material. All of these compositions have a kill rate of 99.9 %. A few claim 99.9 percent kill rate. The overuse of antibiotics over the past twenty years has created a new class of super bacteria and viruses that are very resistant to log 3 antiseptic compounds.

It has been found that these log 3 materials require an exposure time that is longer than the 15 second time period that was claimed ten years ago. This is a serious problem. In trying to achieve higher kill rates with these compositions, one must increase the percentage content of the alcohol. But increasing the content of alcohol has the undesirable effect of increasing the drying rate of the applied antiseptic thereby shortening the time that the applied antiseptic is in the liquid phase. For these compounds to be effective, they must stay in the liquid phase for 15 to 30 seconds. Another problem is that the greater alcohol content increases the incidence of skin irritation.

The following patents are for compositions that are surface disinfectants and incorporate a cationic detergent germicide, (typically alkyl diethyl benzyl ammonium chloride or variants thereof) along with non-anionic or amphoteric detergent stabilizers and scents: U.S. Pat. No. 3,965,026 to Lancz, U.S. Pat. No. 4,464,293 to Dobrin, U.S. Pat. No. 3,932,655 to Conn is directed toward a surgical skin scrub concentrate.

U.S. Pat. Nos. 3,855,140 and 3,960,745 to Bellany et al are also directed to an antibacterial skin scrub and relies on a chemical interaction between the detergent and the composition's major germicidal component, chlorhexidine.

U.S. Pat. No. 3,553,141 to Katsumi, and U.S. Pat. No. 4,576.729to Paszek et al are directed to laundry detergents.

U.S. Pat. No. 5,084,449 discloses use of bisaminophenyl sulfones as sulfur with an alcohol carrier claiming long-term biocidal action.

### SUMMARY OF THE INVENTION

In view of the above, it is an object of this invention to provide a stable microbicidal solution of substantially exclusively hydronium ions that is more effective than presently available solutions of hydronium ions. Such presently available solutions have a pH greater than 2.5.

It is an additional object that the solution be sufficiently stable for commercial purposes.

It is contemplated that versions of this invention be a group of antiseptic compounds that is particularly intended for application on the skin. In this regard it must meet requirements of the twenty first century in the light of the present bioterrorism scare. It is considered that the compound must have a kill efficacy of log 4 or better (99.995 %) and it be effective against new stronger strains of E-Coli 157H7, HIV and hepatitis A.

It is a further object that the antiseptic product of this version invention (for application to the skin) has the following characteristics:

25. fast acting germicidal action:

26. a high degree of germicidal activity against a large variety of microorganisms;

27. non-staining;

28. doesn't irritate the skin with repeated use;

29. Leaves a residue with anti-microbial activity after repeated use so that the product retains long acting potency even after the applied solution has “dried on the skin”.

The version of this invention for application to the skin is directed toward an antiseptic solution having a composition that is primarily activated water with a bactericide and two volatile germicides.

The activated water, which is the basis of this invention, is prepared by a unique procedure for mixing an acid and metal (preferably sulfuric and calcium). to produce an insoluble precipitate that is removed by filtration. The remaining water is left in a state of activation as indicated by a pH that is less than 2.5 in spite of the absence of measurable amounts of cations (e.g., Ca²⁺) or anions (e.g., SO₄₋). While I do not wish to be bound by theory, it is believed that the reduced pH is due to the presence of hydronium ions that renders the water to be a powerful bactericide

This composition, which relies on the activated water as the major solvent, is in contrast to the prior art, which relies on organic liquids as the major solvent.

The actvated water of this invention therefore performs several functions.

1. It is a powerful solvent for the other components of the formulation.

25. It is nonreactive with the skin in comparison to products, which include organic solvents as the major solvent.

3. The use of activated water enables selection from a greater variety of added components than does the use of organic solvents, which serve as a combination bactericide, and solvent in many antiseptic compositions of the prior art. The antiseptic solution of this invention formulated for use on the skin, includes germicides selected from a group of ingredients that includes organic acids, metal salts, quaternary ammonium compounds and alcohols to prolong and improve germicidal strength.

### BRIEF DESCRIPTION OF THE FIGURES

FIG. 1 shows a general method for making solution A.

FIG. 2 shows a method of making solution A using Ca.

### DESCRIPTION OF PREFERRED EMBODIMENTS

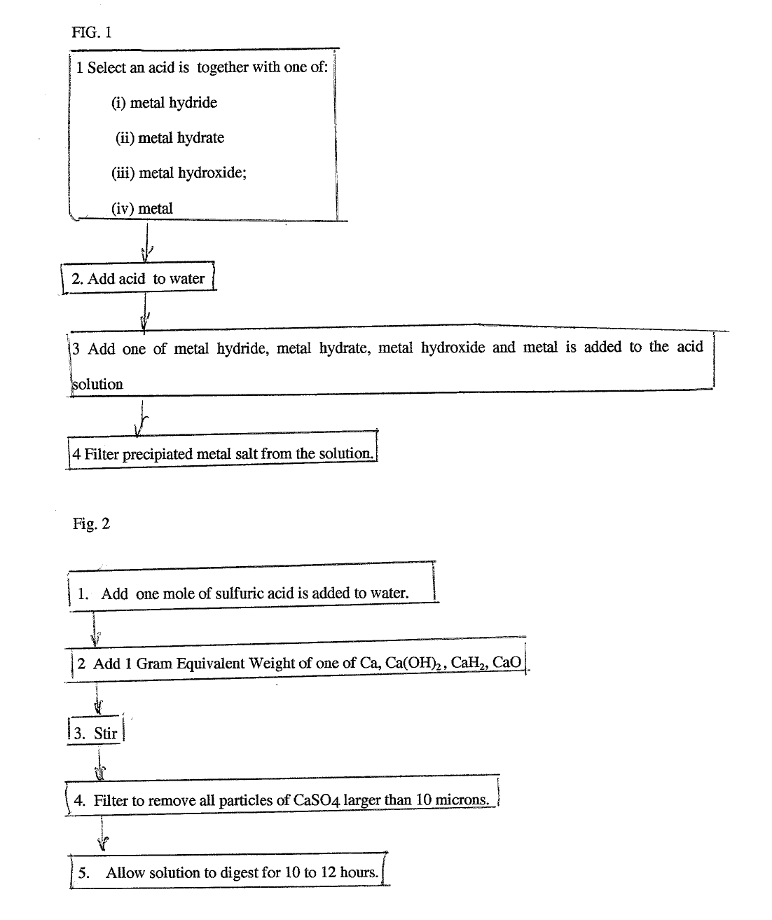

Turning now to a description of the drawings, FIG. 1 is a flow diagram of the method of making the activated water of this solution referred to as solution A.

### PREPARATION OF SOLUTION A

In step 1, an acid is selected together with one of:

(i) metal hydride

(ii) metal hydrate

(iii) metal hydroxide;

(iv) metal

wherein the metal and the anion of the acid form a substantially insoluble salt.

In step 2 the acid is added to water forming an acid having an appropriate concentration.

In step 3 the one of metal hydride, metal hydrate, metal hydroxide and metal is added to the acid solution in a gram equivalent amount equal to the acid leading to the precipitation of the metal salt of the acid.

In step 4, the metal salt is filtered from the solution.

FIG. 2 shows the method in which the metal is calcium and the acid is sulfuric,

In step 1, one mole of concentrated analytic grade of sulfuric acid is added to triple distilled Water.

In step 2, slowly add 1 Gram Equivalent Weight of analytic grade ah₂ to the solution.

In step 3 slowly stir the solution until the reaction is complete producing a new solution.

In step 4 Pass the new solution through a 10 micron filter. removing all particles of CaSO₄ larger than 10 microns.

In step 5, allow the solution to digest for 10 to 12 hours.

In step 6, filter the solution through an 11 micron filter.

To illustrate the invention by way of example, a sample of the invention was prepared in accordance with FIG. 2. Triply distilled water was used and the resultant test sample was found to contain less than 2500 ppm of sulfate and less than 2500 ppm of Calcium. The pH was 2.0. Bactericidal properties of the solution were evaluated by an independent laboratory, BioVir Laboratories, Inc. using the procedure ASTM E1153-87 that is hereby incorporated as reference into this specification. The method was modified in the following manner:

1. 22 mm² coverslips were used for the inoculate step (step 3.2).

2. Only 0.6 ml of ARS-I was used on the coverslips.

3. Trypticase Soy Broth (TSB) pH 10 was employed as pH neutralizer.

4. 10 mL of TSB pH 10 with 0,6 mL of the test sample resulted in a final pH 6.7.

5. The test organisms were Staphylcoccous aureus and Enterobacter aerogenes.

6. Sterile petri dishes were substituted for sterile glass jars.

The results of the test are presented in table I.

| | Expected | | Log | % |
|---|---|---|---|---|
| Organism/Test | (cfu/mL) | Observed | reduction | reduction |
| S. aureus Control | NA | 50,000 | NA | NA |
| S. aureus A | 50,000 | <1 | >4.5 | 99.995 |
| S. aureus B | 50,000 | <1 | >4,5 | 99.995 |
| S. aureus C | 50,000 | 90 | 2.7 | 99.9 |
| E. aerogenes Ctrl | NA | 53,000 | NA | NA |
| E. aerogenes A | 53,000 | <1 | >4.7 | 99.995 |
| E. Aerogenes B | 53,000 | <1 | >4.7 | 99.995 |
| E. Aerogenes C | 53,000 | <1 | >4.7 | 99.995 |

Conclusion of the testing lab (Bio-Vir):

According to these test results, the test sample has demonstrated a capability of a 99.9 ->99.99% bactericidal effect within five minutes.

The pH of the test solution resulting from preparing the test solution in accordance with FIG. 2 was measured to be 1.5 compared 2.7 which was the lowest value reported using the electrolysis method to produce Microwater™ discussed in the BACKGROUND of the specification. The lower the attainable pH, it is expected that the greater would be the bactericidal power of the product.

The reaction of the solution of this invention on human tissue has been tested many times in this laboratory at pH 2.0 and it has been found that there is no reaction whatsoever with periods of fifteen minutes exposure.

Solution A is a general antiseptic solution of this invention and serves as a basic ingredient in various versions of the invention designed for specific uses. These specific uses include application to human skin for which Examples 1 and 2 (discussed below) are intended.

Sterilizing the surface of human tissue (skin) poses additional problems that are addressed by additional versions of the antiseptic solution of this invention.

One problem is to maintain a residual sterilizing capability after the original solvent (activated water) has evaporated. This action is accomplished according to one version of this invention by incorporating a “carrier” into the antiseptic solution that is retained as a film on the treated surface and retains active bactericidal constituents after the activated water has evaporated. Retention of these constituents prevents recontamination of the surface.

Mineral oil and glycerol are preferred carriers for the purposes of this invention. However, an emulsifying agent must be mixed with the carrier in order to achieve intimate emulsification of the carrier in the activated water. A preferred emulsifying agent is alcohol. A successful practice is to mix the carrier in the alcohol BEFORE adding it to the activated water in order that the carrier is sufficiently dispersed.

Inclusion of quaternary ammonium compounds is a practice of this invention for two reasons.

One reason is that such compounds are powerful antiseptics.

A second reason is that quaternary ammonium compounds are hygroscopic so that the moisture dependent antiseptic characteristics of the film that depend on the moisture content of the film are retained for a longer period with the antiseptic solution of this invention containing these compounds.

Table 1 lists a preferred composition of an antiseptic solution according to the invention for sterilizing human tissue. (the skin)

TABLE 1

| INGREDIENT | PERCENT BY WEIGHT |
|---|---|
| 1. Water | 60-70 |
| 2 Isopropanol | 3-5 |
| 3. Ethanol | 18-25 |
| 4. Lactic Acid | ½-1 |
| 5. Calcium Chloride | ½-1 |
| 6.. Dimethyl Benzyl Ammonium Chloride | ½-1 |
| 7. Mineral Oil | 1-2 |
| 8. Solution A | ½-1 |
| 9. Natural fragrance | |

Solution A produced by this procedure consists essentially of less than 2500 parts per million of calcium sulfate, and has a pH of less than 2.5 wherein pH of less than 2.5 is maintained for longer than 48 hours.

According to the invention, a method is disclosed for creating a solution containing a working concentration of hydronium ions which kill the bacteria apparently by robbing the oxygen from the bacteria. The unique disinfecting solution is prepared by a process in which calcium sulfate is generated and then completely filtered out so the concentration of either Calcium or sulfate is less than 2500 ppm. Calcium Sulfate is very insoluble so that, by mixing stoichiometric ratios of sulfuring acid and Ca Hydride, and then filtering with a ten micron filter, the level of calcium sulfate left in the water is less than tap water purity.

The following examples illustrate the method of preparation of the antiseptic product of this invention for application to the skin.

### EXAMPLE 1

1. Place 30 gallons of water in a first mixing tank.

2. Add one gallon of solution A.and mix for five minutes.

Add:

three pounds of lactic acid,

two pounds of calcium chloride

one liter of dimethyl benzyl ammonium chloride

In any order and mix thoroughly.

3. Mix 2 gallons of mineral oil in twenty five gallons of ethanol and five gallons of isopropanol in a second tank.

4. Mix 25 gallons of water with the contents of the second tank.

5. Mix the contents of the first mixing tank with the contents of the second mixing tank

6. If the pH of the mixture is greater than 2.0, add sufficient solution A to reduce the pH to 1.8.

A sample of the invention was prepared in accordance with FIG. 2. The pH was less man 2,0. Bactericidal properties were evaluated by BioVir Laboratories, an independent laboratory. Using the procedure ASTM E1153-87 (hereby incorporated as reference into this specification. The method was modified in the following manner.

1. Oversleeps (22 mm²) were used for the inocula step (step 3.2)

2. 0.6 ml of solution A was applied to the coverslips.

3. Tryticase Soy Broth (TSB) pH 10 was employed as a pH neutralizer

4. Test organisms being Staphylococcus aureus and Enterobacter aerogenes were provided.

5. Sterile petri dishes were substituted for stile glass jars.

Results of the test are presented in Table 2.

TABLE 2

| | INITIAL | FINAL | | |
|---|---|---|---|---|
| ORGANISM | (cfu/ml) | (cfu/ml) | log change | % reduction |
| S aureus control | NA | 50,000 | NA | NA |
| S aureus A | 50,000 | <1 | >4.5 | 99.995 |
| S aureus B | 50,000 | <1 | >4.5 | 99.995 |
| S aureus C | 50,000 | 90 | 2.7 | 99.9 |
| E. aerogenes Ctrl | NA | 53,000 | NA | NA |
| E. aerogenes A | 53,000 | <1 | <4.7 | 99.995 |
| E aerogenes B | 53,000 | <1 | <4.7 | 99.995 |
| E. aerogenes C | 53,000 | <1 | <4.7 | 99.995 |

Direct quotation of the Conclusion of the BIOVIR TESTING LABORATORY: “THE TEST SAMPLES HAVE DEMONSTRAED A CAPABILITY OF A 99.995+% BACTERICIDAL EFFECT WITHIN FIVE MINUTES. THE LOWER THE SUSTAINABLE pH, THE GREATER IS THE BACTERICIDAL POWER OF THE PRODUCT.

The bactericidal action of this invention has been tested many times in this laboratory at pH 2.0 or below and it has been found that there is no adverse skin reaction whatsoever with periods of exposure of fifteen minutes and longer.

### EXAMPLE 2

A second formulation is presented for people with sensitive skin. The second formulation is prepared according to the following steps:

1. Place 30 gallons of water in a first tank.

1. Add one gallon of solution A

2. Mix for five min.

3. Add:

3 lb. Lactic acid

b 2 lb calcium chloride

1 liter of dimethyl benzyl ammonium chloride

mix thoroughly

4. Mix in a second tank:

18 gallons of ethanol

2 gallons of isopropanol

1 gallon glycerol

mix thoroughly

5. Mix 35 gallon of water into the second tank

26. Mix the contents of the second tank and the first tank and stir.

7. If the pH is above 2.0, add sufficient solution A to reduce the pH to not more than 1.8.

The antiseptic solution produced in accordance with procedures of EXAMPLE 2 has been determined to have a bactericidal strength about as strong as EXAMPLE 1.

An important difference between the solution of example 1 and example 2 is that the percent water content is greater in sample 2, i.e., the alcohol content (isopropanol and ethanol) is reduced Increasing the percent water content has two important effects:

1. The drying time of the antiseptic applied to the surface is lengthened thereby increasing the bactericidal effect.

2. The reduction of alcohol content provides a solution that is more tolerable to sensitive skin.

An important feature of this invention is the inclusion of solution A in the formulation. The inclusion of Solution A is a novel formulation. While I do not wish to be bound by theory, it's believed that bactericidal properties are derived from the presence of hydronium ions.

The bactericidal strength of the antiseptic solution of this invention is increased by reducing the pH by the addition of solution A. Solution A, added in an appropriate amount reduces the pH of the antiseptic solution to 1.5, providing an antiseptic solution that does not “burn” skin that is exposed to the skin for a period of time sufficient for effective bactericidal reaction (a log 5 kill).

The organic acid, (preferably lactic or oxalic) is included as a feature of this invention because it has a low dissociation constant and therefore maintains the low pH of the solution thereby extending the life of the bactericidal characteristic.

A quaternary ammonium compound such as diethyl benzyl ammonium chloride is a low volatility secondary germicide. Quaternary ammonium compounds are also hygroscopic. The novel combination of the quaternary ammonium compound with solvent A as disclosed by this invention reduces the evaporation rate of the water applied to the skin so that residual bactericidal properties are prolonged.

A metel salt as calcium chloride or silver chloride was selected for its long lasting bacteriological effect. The carrier, one of mineral oil and Glycerol, leave a film after the activated water has evaporated that retains the these metal salts and prolongs the bactericidal environment (including the mineral salts) of the area (skin).

Just sufficient alcohol is included to ensure uniform dispersal of the carrier in the activated water.

Variations and modifications may be contemplated that apply the principles of this invention and are within the scope of the invention.

For example, dimethyl benzyl ammonium chloride has been disclosed as a preferred quaternary ammonium compound because of its hygroscopic and bactericidal characteristics. However, other quaternary ammonium chlorides may be used which have similar properties. An alternative quaternary ammonium compound for this appellation is selectable from the group of compounds that consists of diethyl benzyl ammonium chloride, benzalkonium chloride, diethyl dodecyl benzyl ammonium chloride, dimethyl didodecyl ammonium chloride, octadecyl dimethyl benzyl ammonium chloride, trimethyl tetradecyl ammonium chloridem, trimethyl octadecylammonium chloride, trimethyl hexadecyl ammonium chloride, Alkyl dimethyl benzyl ammonium chloride, cetyl pyridinium bromide, cetyl pyridinium chloride, dodecylpyridinium chloride, and benzyl dodecyl bis(B-hydroxyethyl ammonium chloride.

Calcium Chloride has been included as a preferred metal in the solution because of its bactericidal characteristic and because of its solubility in the antiseptic solution and its capability to remain in the film formed by the residual carrier on the skin after the water and alcohol have evaporated. On skilled in the art will recognize that other metal salts would also be effective for this purpose and include. These metal salts are selectable from a group of metal salts having any one of a number of cations and any one of a number of anions.

The metals generally fall in the range designated on the periodic chart as 1(A,B), II(A,B) III (A), IV (A, B), VI B, VII. This group includes rare earth compounds.

For example, list of metal salts used in place of or along with Ca Cl₂ would include of tin chloride, tin nitrate, tin acetate, tin bromide, tin iodide, iron chloride, iron nitrate, iron acetate, iron bromide, iron iodide, iron nitrate, iron acetate, iron bromide, iron iodide. magnesium chloride, magnesium nitrate, magnesium acetate, magnesium bromide, magnesium iodide, copper sulfate, copper chloride, copper nitrate, copper acetate, copper bromide, copper iodide.

Other organic acids may substitute for lactic acid including oxalic acid.

The environments in which the antiseptic solution is is to be applied vary widely from one another. Such circumstances include variations of temperature, humidity, species of bacteria, as well as characteristics of the individual, particularly variations in allergic response. It is therefore submitted that, although the specific examples presented in this specification are useful for a wide range of these conditions, it is nevertheless contemplated that the content expressed as percent volume of the metal salts, organic acids, quaternary ammonium compound, will be selected from a broad range from .01 % to 20 % by volume.

The fragrance of an antiseptic solution applied to human skin is an important factor in establishing the marketability of the product. Substances that have been found to confer an agreeable fragrance to the antiseptic solution include vanilla extract in alcohol and eucalyptus oil.

In view of these variations and modifications that are within the scope of the invention, it is therefore wished to define the scope of the invention by the appended claims.

What is claimed is:

1. A method for preparing a solution having disinfectant properties which comprises the steps in operable order
(a) Selecting one of a metal, a metal hydride a metal oxide and a metal hydroxide and an acid having an anion capable of forming an insoluble salt with said metal;
(b) mixing a gram molar quantity of said acid in water;
(c) Stirring into said acid in water an equivalent of said gram molar quantity of said metal in one of said metal, metal hydride, metal oxide and metal hydroxide;
(d) Passing the resultant solution of step (c) through a filter to remove precipitates of salt formed by said metal and said acid.

2. The method of claim 1 wherein said step (a) includes the step:
selecting said metal to be calcium and said acid to be sulfuric acid.

3. A solution consisting essentially of less than 2500 parts per million of calcium sulfate, and a pH of less than 2.5.

4. A solution consisting essentially of less than 2500 parts per million of calcium sulfate, sufficient hydronium ions to provide a pH of less than 2.5 wherein pH of less than 2.5 is maintained for longer than 48 hours.

5. A method for preparing a solution having a pH of less than 2.5, and less than 2500 parts per million of calcium sulfate which comprises the steps in operable combination of:
(a) forming a solution of one mole H₂SO₄ per one liter of water:
(b) stirring into said solution of step (a) one Gram Equivalent Weight of one of CaH_{2,} Ca, CaO, Ca(OH)₂ per one mole of H₂SO₄
(c) filtering the solution of step (b) through an eleven micron filter;
(d) allowing the solution of step (c) to digest for at least 10 hours;
(e) filtering the solution of step (d) through a two micron filter.

6. A method for treating an infected area of a human which includes the steps:
(a) forming a solution of one mole H₂SO₄ per one liter of water:
(b) stirring into said solution of step (a) one Gram Equivalent Weight of one of CaH_{2,} Ca, Ca(OH)/CaO per one mole of H₂SO₄
(c) filtering the solution of step (b) through an eleven micron filter;
(d) allowing the solution of step (c) to digest for at least 10 hours;
(e) filtering the solution of step (d) through a two micron filter providing a disnfecting solution consisting of less than 2500 ppm Ca⁺⁺ and SO₄⁻⁻ and a pH less than 2.5 that is stable longer than 48 hours;
(f) applying said disinfecting solution to said infected area

7, An antiseptic solution for sterilizing a surface of skin of a human which comprises:
water;
said solution of claim 1 added to said water in sufficient concentration to reduce pH to below 2.5;
at least one of oxalic acid, lactic acid and quaternary ammonium;
a metal salt selected to have a bactericidal characteristic;
a carrier for retaining said metal salt and said at least one of oxalic acid, lactic acid and quaternary ammonium chloride in a film of said carrier;
an organic solvent;
said organic solvent selected in sufficient concentration to one of emulsify and dissolve said carrier and selected to have a vapor pressure that is substantially greater than a vapor pressure of said carrier whereby a film of said carrier retaining said metal salt and said at least one of oxalic acid and lactic acid and said quaternary ammonium compound is left as an antiseptic residue after said organic solvent and water have evaporated.

8. The antiseptic solution of claim 7 wherein said organic solvent is at least one of ethanol and isopropanol.

9. The antiseptic solution of claim 7 wherein the content of said lactic acid in said antiseptic solution is selected from a range between 0.01 and 20 percent by volume.

10. The antiseptic solution of claim 7 wherein the content of said oxalic acid in said antiseptic solution is selected from a range between 0.01 and 20 percent by volume.

11. The antiseptic solution of claim 7 wherein the content of said metal salt in said antiseptic solution is selected from a range between 0.01 and 20 percent by volume.

12. The antiseptic solution of claim 7 wherein the content of said quaternary ammonium compound in said antiseptic solution is selected from a range between 0.01 and 20 percent by volume.

13. The composition of claim 7 wherein said quaternary ammonium compound is selected from a group of compounds that consists of diethyl benzyl ammonium chloride, benzalkonium chloride , diethyl dodecyl benzyl ammonium chloride, dimethyl didodecyl ammonium chloride, octadecyl dimethyl benzyl ammonium chloride, trimethyl tetradecyl ammonium chloridem, trimethyl octadecylammonium chloride, trimethyl hexadecyl ammonium chloride, Alkyl dimethyl benzyl ammonium chloride, cetyl pyridinium bromide, cetyl pyridinium chloride, dodecylpyridinium chloride, and benzyl dodecyl bis(B-hydroxyethyl ammonium chloride.

14. The antiseptic solution of claim 7 wherein a pH of said solution is adjust to a value in arrange between 15 and 1.8 by additions of the solution of claim 1.

15. The antiseptic solution of claim 7 wherein a metal of said metal salt is selected from a group of metals identified as 1(A,B), II(A,B) III(A), IV(A, B), VI B, VII rare earth compounds and combinations thereof.

16. The composition of claim 7 wherein a metal of said metal salt is selected from a group of metals that consists of calcium, magnesium, tin, iron, copper, silver.

17. The composition of claim 7 wherein said metal salt is a combination of metal salts and each metal of said combination is selected from a group of metals that consists of calcium, magnesium, tin, iron, copper, silver.

18. The antiseptic solution of claim 7 wherein said metal salt is selected from a group of metal salts which consists of at least one of copper sulfate, copper chloride, copper nitrate, copper acetate, copper bromide, copper iodide.

1. The antiseptic solution of claim 7 wherein said metal salt is selected from a group of metal salts which consists of at least one of tin chloride, tin nitrate, tin acetate, tin bromide, tin iodide.

20. The antiseptic solution of claim 7 wherein said metal salt is selected from a group of metal salts which consists of at least one of iron chloride, iron nitrate, iron acetate, iron bromide, iron iodide.

21, The antiseptic solution of claim 7 wherein said metal salt is selected from a group of metal salts which consists of at least one of calcium chloride, iron nitrate, iron acetate, iron bromide, iron iodide.

22, The antiseptic solution of claim 7 wherein said metal salt is selected from a group of metal salts which consists of at least one of magnesium chloride, magnesium nitrate, Magnesium acetate, magnesium bromide, magnesium iodide.

23. The antiseptic solution of claim 7 wherein said carrier is mineral oil.

24. The antiseptic solution of claim 7 wherein said carrier is glycerine.

25 The antiseptic solution of claim 7 which further comprises a fragrance.

26. The antiseptic solution of claim 25 wherein said fragrance is vanilla extract in alcohol.

27. The antiseptic solution of claim 25 wherein said fragrance is eucalyptus oil.
